(19) United States
(12) Plant Patent Application Publication
(10) Pub. No.: US 2015/0282417 P1
(43) Pub. Date: Oct. 1, 2015

(54) Sutera plant named 'DANCOP103'

(50) ;

(71) Applicant: Gavriel Danziger, Moshav Mishmar Hashiva (IL)

(72) Inventor: Gavriel Danziger, Moshav Mishmar Hashiva (IL)

(21) Appl. No.: 13/999,860

(22) Filed: Mar. 28, 2014

(51) Int. Cl. A01H 5/00

(52) U.S. Cl. PLT/485

(57) ABSTRACT

A new and distinct Sutera cultivar named ‘DANCOP103’ is disclosed, characterized by early and abundant extra large white flowers, a mounded, very compact habit, very well branched form and medium vigor. The new variety is a Sutera, normally produced as an outdoor garden or container plant.

### LATIN NAME OF THE GENUS AND SPECIES

Sutera cordata

### VARIETY DENOMINATION

‘DANCOP103’

### BACKGROUND OF THE INVENTION

The new Sutera cultivar is a product of a planned breeding program conducted by the inventor, Gavriel Danziger, in Moshav Mishmar Hashiva, Israel. The objective of the breeding program was to produce new Sutera varieties for ornamental commercial applications. The cross resulting in this new variety was made during March of 2009.

The seed parent is the unpatented, proprietary seedling variety referred to as Sutera cordata ‘cv. 09-1202’ The pollen parent is unknown as the new variety is the result of an open pollination breeding program. The new variety was discovered in November of 2010 by the inventor in a group of seedlings resulting from the 2009 crossing, in a research greenhouse in Moshav Mishmar Hashiva, Israel.

Asexual reproduction of the new cultivar was performed by vegetative cuttings. This was first performed at a research greenhouse in Moshav Mishmar Hashiva, Israel during December of 2010 and has shown that the unique features of this cultivar are stable and reproduced true to type in more than 20 successive generations.

### SUMMARY OF THE INVENTION

The cultivar ‘DANCOP103’ has not been observed under all possible environmental conditions. The phenotype may vary somewhat with variations in environment such as temperature, day length, and light intensity. without, however, any variance in genotype.

The following traits have been repeatedly observed and are determined to be the unique characteristics of ‘DANCOP103’ These characteristics in combination distinguish ‘DANCOP103’ as a new and distinct Sutera cultivar:

1. Mounded, very compact growth habit.

2. Extra large white flowers.

3. Very branched.

4. Early, abundant flowering.

5. Medium vigor.

Plants of the new cultivar ‘DANCOP103’ are similar to plants of the seed parent, Sutera cordata ‘cv. 09-1202’ in most horticultural characteristics, however, plants of the new cultivar ‘DANCOP103’ differs in the following ways:

1. Parent variety has a semi-trailing, elongated growth habit.

2. Parent variety has smaller white flowers.

3. Seed parent has less branching.

4. Seed parent begins flowering late, with a medium quantity flowering.

5. Parent variety grows more vigorously.

### COMMERCIAL COMPARISON

Plants of the new cultivar ‘DANCOP103’ are comparable to the commercial variety Sutera ‘DANCOP25’ U.S. Plant Pat. No. 19,747. The two Sutera varieties are similar in most horticultural characteristics; however, the new variety ‘DANCOP103’ differs in the following ways:

1. Comparison variety has elongated, semi-trailing growth habit whereas candidate variety has mounded, very compact growth habit.

2. Comparison variety has white flowers, larger than those of the new variety.

3. Comparison variety has medium branching whereas candidate variety is very well branched.

4. Comparison variety plant height is shorter than the height of the new variety.

5. Comparison variety has strong vigor whereas candidate variety has medium vigor.

Plants of the new cultivar ‘DANCOP103’ are also comparable to the unpatented commercial variety Sutera ‘Scopia Great White improved’. The two Sutera varieties are similar in most horticultural characteristics; however, the new variety ‘DANCOP103’ differs in the following ways:

1. Comparison variety has semi-trailing, elongated growth habit whereas candidate variety has mounded, very compact growth habit.

2. Comparison variety has large, white flowers whereas candidate variety has extra-large flowers.

3 Candidate variety is more branched than comparison variety.

4. Comparison variety does not bloom as early as candidate variety, and flowering of comparison variety is not as abundant as candidate variety.

5. Comparison variety has strong vigor whereas candidate variety has medium vigor.

### BRIEF DESCRIPTION OF THE PHOTOGRAPHS

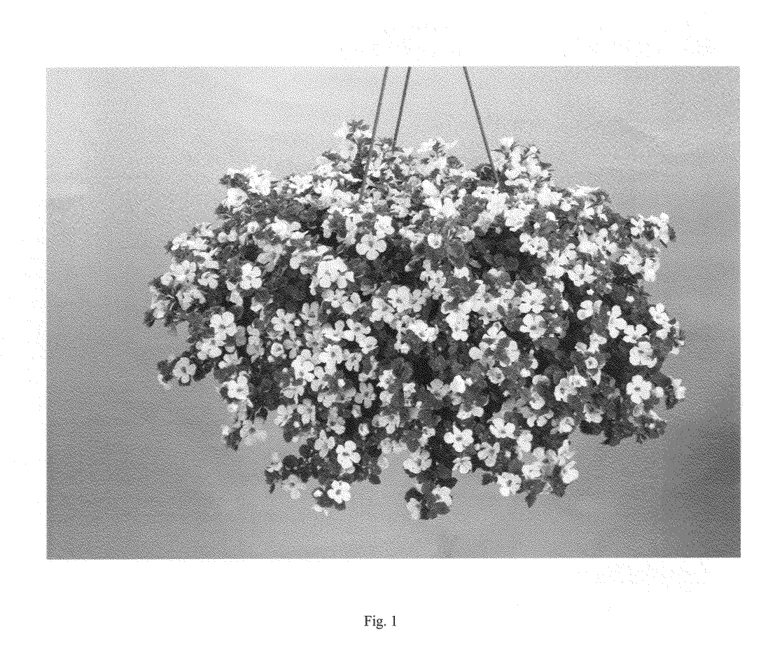

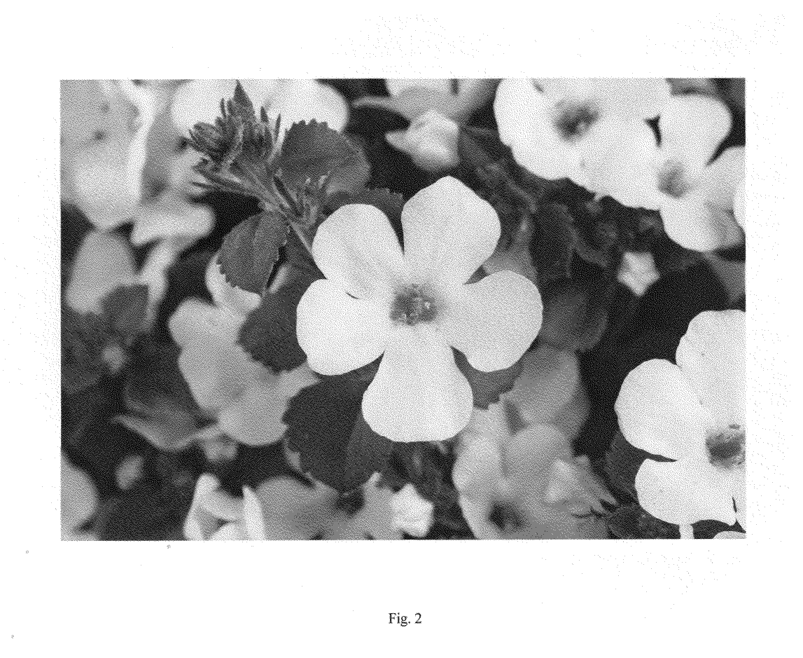

The accompanying photograph in FIG. 1 illustrates in full color a typical plant of ‘DANCOP103’ grown in a greenhouse, in a 13 cm pot. Age of the plant photographed is approximately 3 months old from rooted cutting. FIG. 2 illustrates a close up of the flowers. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Mini Colour Chart 2005 except where general terms of ordinary dictionary significance are used. The following observations and measurements describe ‘DANCOP103’ plants grown in a greenhouse during Winter to Spring in Moshav Mishmar Hashiva, Israel. The growing temperature ranged from 15° C. to 26° C. during the day and from 13° C. to 16° C. during the night. Measurements and numerical values represent averages of typical plant types.
Botanical classification: Sutera cordata ‘DANCOP103’
Propagation:
   Typical propagation material.—Vegetative cuttings.
   Time to rooting.—7 to 10 days at approximately 20° C.
   Root description.—Fine, fibrous roots.
Plant:
   Age of plant described.—Approximately 3 months old.
   Pot size of plant described.—13 cm.
   Height.—about 16 cm.
   Plant spread.—About 45 cm.
   Growth rate.—Medium.
   Branching characteristics.—
   Length of primary lateral branches.—About 20 cm.
   Diameter of lateral branches.—About 0.2 cm.
   Quantity of primary lateral branches.—About 12.
   Characteristics of primary lateral branches.—Form: Cylindrical Diameter: About 0.2 cm. Color: RHS Green 137 B Texture: Velvety Strength: Strong.
   Internode length.—About 1.5 cm.
Foliage:
   Leaf.—Arrangement: Opposite, petiolate Quantity: Approximately 40 per branch. Average Length: 2.5 cm. Average Width: 2.2 cm. Shape of blade: Ovate Apex: Obtuse Base: Acuminate Margin: Serrate Texture of top surface: Velvety. Slightly pubescent Texture of bottom surface: Velvety. Slightly pubescent Aspect: About 90° to the branch Color: Young foliage upper side: RHS Green 137 C Young foliage under side: RHS Green 137 D Mature foliage upper side: RHS Green 137 A Mature foliage under side: RHS Green 137 B Venation: Type: Pinnate. Venation color upper side: RHS Green 137 A Venation color under side: RHS Green 137 B Petiole: Length: About 0.3 cm. Diameter: About 0.2 cm. Color: RHS Green 137 A Texture: Slightly velvety.
Flower:
   Natural flowering season.—All year round.
   Days to flowering from rooted cutting.—About 45.
   Inflorescence and flower type and habit.—Axillary, solitary salverform single flowers.
   Rate of flower opening.—3 to 5 days from bud to fully opened flower.
   Flower longevity on plant.—About 7 days.
   Persistent or self-cleaning.—Persistent.
   Bud.—Shape: Spatulate Length: About 1.6 cm Diameter: About 0.7 cm Color: RHS White 155 C.
   Flower size.—Diameter: About 2.6 cm. Length: About 1.5 cm. Corolla tube diameter: About 0.4 cm.
   Corolla/petals.—Arrangement: Symmetric Lobe Length: About 1.0 cm. Lobe Width: About 0.9 cm. Quantity: 5 Texture: Velvety Lobe Apex: Rounded Lobe shape: Rounded Margin: Entire Aspect: About 90° to the tube.
   Color.—When opening Upper surface: RHS White 155 C Lower surface: RHS White 155 C Fully opened: Upper surface: RHS. White 155 C Lower surface: RHS White 155 C Visible Corolla tube color: RHS Yellow 13 A.
   Calyx/sepals.—Quantity per flower: 5 Shape: Linear Length: About 0.6 cm Width: About 0.1 cm Apex: Acute Base: Fused. Margin: Entire Texture: Slightly velvety Color: Upper Surface: RHS Green 137 B Lower Surface: RHS Green 137 C.
   Peduncle.—None.
   Pedicel.—Length: Approximately 1.0 cm. Diameter: Approximately 0.1 cm. Color: Near RHS Green 137 C Orientation: Slanted upward and outward Pubescence: Slightly pubescent.
   Fragrance.—None.
Reproductive organs:
   Stamens.—Number: 4 Filament length: Approximately 1 cm.
   Anthers.—Shape: Cylindrical Length: Approximately 0.1 cm. Color: RHS Yellow 13 B Pollen: Color: RHS Yellow-Orange 17 B Quantity: Sparse.
   Pistil.—Number: 1 Length: About 1.1 cm. Style: Length: About 0.9 cm. Color: RHS Yellow-Green 145 B Stigma: Shape: Undulate Color: RHS Yellow-Green 146 C Ovary Color: RHS Green 143 B.
Other characteristics:
   Seeds and fruits.—About 20 elliptic, small (1 mm long) light brown seeds per one elliptic Brown capsule. Seed color unmeasurable because of small size. Capsules near Brown 200D.
   Disease/pest resistance.—Typical to known Sutera varieties.
   Temperature tolerance.—The new variety tolerates temperatures between 5 to 40° C.

What is claimed is:

1. A new and distinct cultivar of Sutera plant named ‘DANCOP103’ as herein illustrated and described.
